(19) United States
(12) Patent Application Publication
(10) Pub. No.: US 2007/0158513 A1
(43) Pub. Date: Jul. 12, 2007

(54) Surgical clamp and tool support system

(75) Inventors: Steven LeVahn, Lino Lakes, MN (US); Todd M Bjork, River Falls, WI (US); Ronald Von Wald, Centerville, MN (US); Todd W. Sharratt, Stillwater, MN (US)

Correspondence Address: SHEWCHUK IP SERVICES, 533 77TH STREET WEST, EAGAN, MN, 55121, US

(73) Assignee: LeVahn Intellectual Property Holding Company, LLC, St. Paul, MN (US)

(21) Appl. No.: 11/330,625

(22) Filed: Jan. 12, 2006

(51) Int. Cl. G09F 7/18; A61B 1/32

(52) U.S. Cl. 248/229.21; 600/228; 600/234

(57) ABSTRACT

A surgical clamp includes a support frame clamp member, a retraction shaft clamp member and a handle linked to the support frame clamp member and the retraction shaft clamp member for moving the clamp members between loosened and tightened positions. The shaft clamp and the support frame permit pivoting of the retraction shaft relative to the support frame. The handle is disposed to move through a throw of less than 90°, always outside the support frame and always in alignment with the retractor shaft. For a standard orientation of the clamp, the tightening throw is vertically upward, on a handle which is balanced and aligned relative to the pivot axis and possibly relative to the connection of the frame clamp to the support frame.

### CROSS-REFERENCE TO RELATED APPLICATION(S)

None.

### BACKGROUND OF THE INVENTION

The present invention relates to the field of surgical tools, and particularly to the design and manufacture of surgical retractor systems. Surgical retractor systems are used during surgery to bias and hold tissue in a desired position. As one example, some surgical procedures require anterior access to the spine, through the patient's abdomen. Tissue such as skin, muscle, fatty tissue and interior organs needs to be held retracted to the side so the surgeon can obtain better access to the vertebrae structures of primary interest.

Surgical retraction may be performed by one or more aides using handheld tools, with the most basic retractor apparatus being a tongue depressor. More commonly now in sophisticated operating rooms during abdominal or chest surgery, a surgical retractor system or assembly is used. The retractor assembly may, for instance, include a ring or support frame which is rigidly supported from the patient's bed above and around the surgical incision location, with a number of clamps and retractor blades to hold back tissue proximate to the surgical incision. Other retraction systems, such as those disclosed in U.S. Pat. Nos. 6,315,718, 6,368,271 and 6,659,944 to Sharratt, incorporated herein by reference, may not include a ring and/or may be directed at other types of surgery. Clamps may also be used to attach the ring or support frame to a support post 22 and/or part of the bed frame.

In devising a proper clamping structure, the clamp should give the surgeon flexibility in quickly assembling the retraction system and in placement of the various retractors. Once the various retractors are in place and oriented and pulled as desired, the retraction system clamps should allow quick and easy tightening so the entire retraction system is maintained fixedly in place. Once tightened the retraction system should be unobtrusive so neither the tissue held retracted nor the retraction system interfere in any way with the surgeon or the surgical procedure. After surgery is completed (or perhaps once or more during surgery), the retraction system should-quickly loosen and/or disassemble so as relax the retracted tissue and minimize damage to the retracted tissue. Surgical retractor systems must be robust and strong, as even a slight possibility of failure during use is not tolerated. Surgical retractor assemblies should be readily reusable, including sterilizable, for use in multiple surgeries. Surgical retractor systems should maintain a relatively low cost. Improvements in surgical retractor clamps and systems can be made in keeping with these goals.

### BRIEF SUMMARY OF THE INVENTION

The present invention is a clamp and a support system using the clamp. The clamp has a first clamping opening for receiving the support frame and second clamp opening for receiving the retractor or tool shaft. A handle controls operation of at least one of the clamp members. In one aspect, the retractor or tool shaft can be pivoted relative to the support frame, and the handle follows this pivot and always resides vertically under the shaft. In another aspect, the handle can be moved from a loosened position to a tightened position with a balanced, single hand application of squeezing force, similar to operation of a scissors. In another aspect the clamp is very low profile and positioned for minimal interference with the surgical arena.

### BRIEF DESCRIPTION OF THE DRAWINGS

FIG. 1 is a perspective view of a prior art surgical retractor system showing clamp location and use during surgery.

FIG. 2 is a perspective view of a preferred surgical clamp in accordance with the present invention.

FIG. 3 is an elevational view of the clamp of FIGS. 1 and 2, showing the tightened position of the handle in dotted lines.

FIG. 4 is a plan view of the clamp of FIGS. 2 and 3, showing the range of adjustment of the shaft and handle relative to the support frame.

FIG. 5 is an enlarged cross-sectional view of a portion of the clamp taken along lines 5-5 in FIG. 4, showing the tightened position of the clamp in dotted lines.

FIG. 6 is an enlarged cross-sectional view of the portion of the clamp of FIG. 5, but shown in the tightened position, showing the loosened position of the clamp body in dotted lines.

FIG. 7 is an end view of the preferred clamp of FIGS. 2-6 in the loosened position.

FIG. 8 is an end view of the preferred clamp similar to FIG. 7, but in the tightened position.

While the above-identified drawing figures set forth one or more preferred embodiments, other embodiments of the present invention are also contemplated, some of which are noted in the discussion, In all cases, this disclosure presents the illustrated embodiments of the present invention by way of representation and not limitation. Numerous other minor modifications and embodiments can be devised by those skilled in the art which fall within the scope and spirit of the principles of this invention.

### DETAILED DESCRIPTION

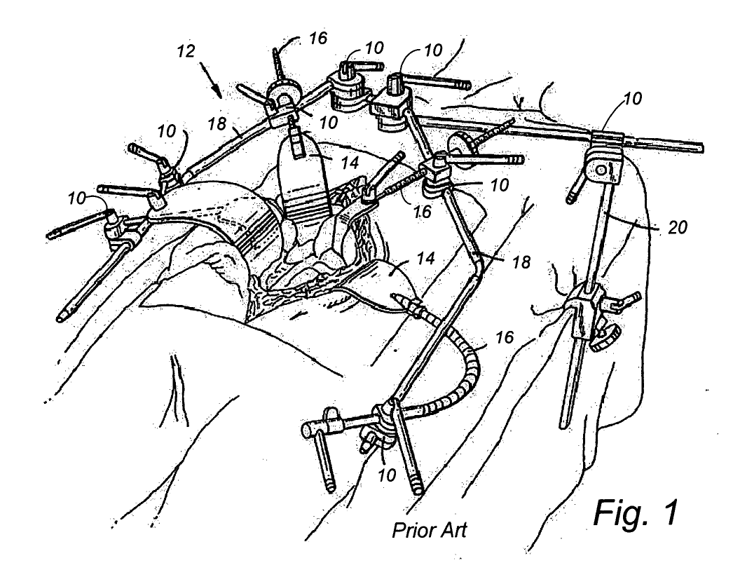

As shown in FIG. 1, one or more prior art clamps 10 are used in a surgical retraction system 12. The surgical retraction system 12 is used to support several surgical retraction blades 14 during surgery. The retractor blades 14 may be in accordance with prior art systems, or in accordance with those disclosed in pending application Ser. No. 11/247,817 filed Oct. 11, 2005, assigned to the assignee of the present invention and incorporated by reference. Each retractor blade 14 is attached to a retractor blade shaft 16. Each retractor blade shaft 16 is clamped with a clamp 10 to a retractor ring or support frame 18. The clamps 10 permit the surgeon to adjust the horizontal location (in and out) as well as the angular orientation of the shafts 16 and then permit the surgeon to securely fasten each retractor blade 14 once a desired position and orientation is achieved. The retractor ring 18 is supported relative to the patient's bed, such as by clamping to one or more retractor posts 20, and one or more clamps 10 can be used in this location as well. Each of the retractor blade shaft 16, the retractor ring 18 and the retractor post 22 may be, formed primarily of a metal material such as surgical stainless steel as known in the surgical retractor art. In addition to the retractor components shown, clamps 10 can also or alternatively be used to clamp surgical tools or other apparatus (not shown), such as a camera, a light or a catheter to a support structure or together.

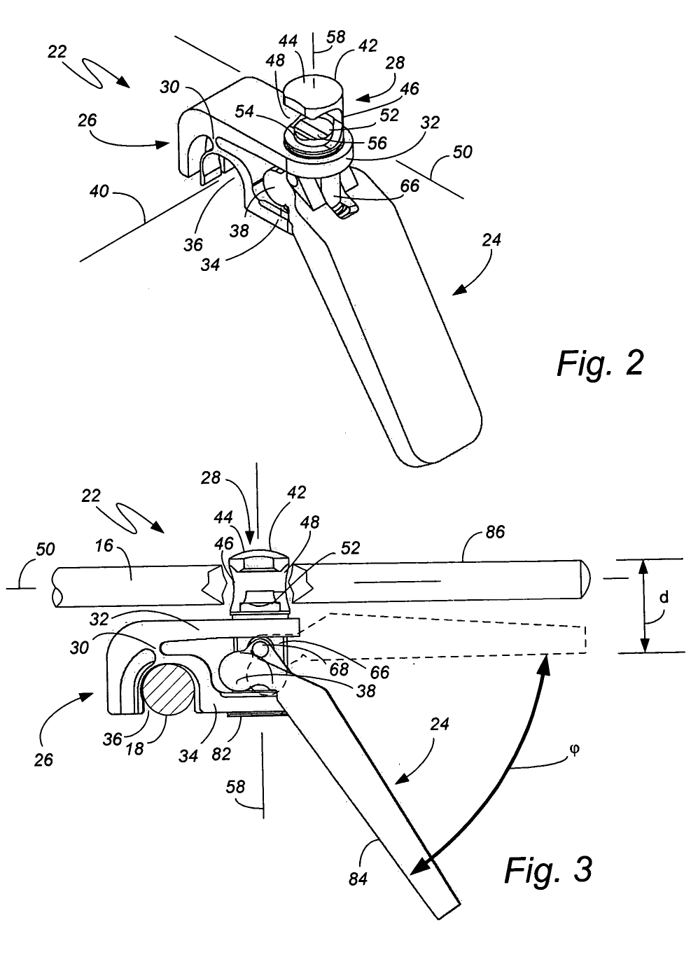

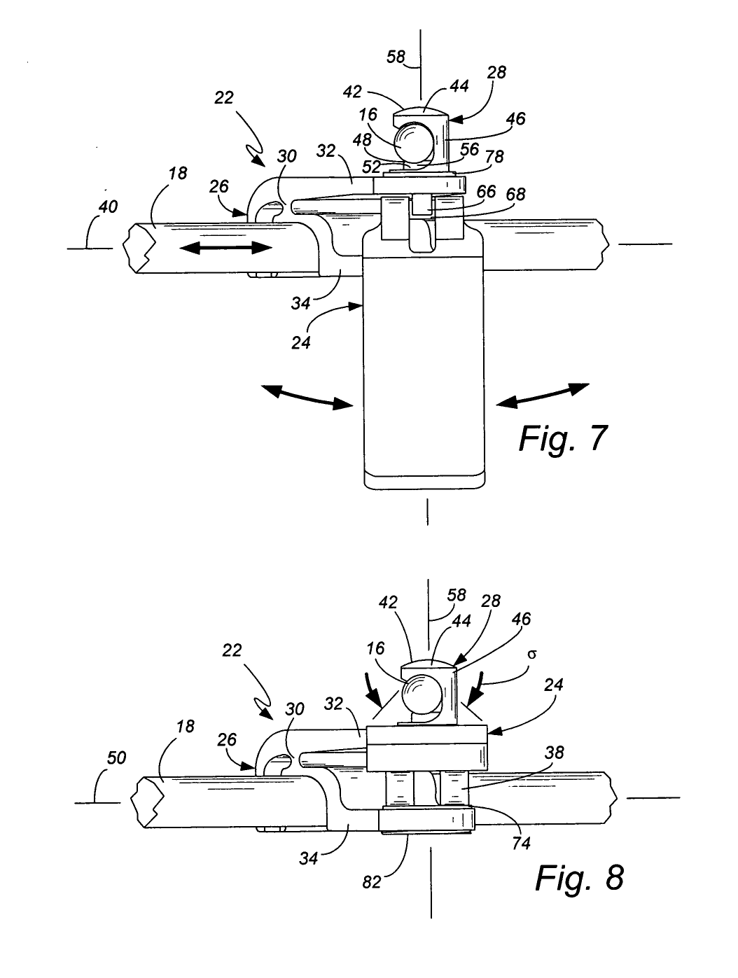

The clamp 22 of the present invention shown in FIGS. 2-8 can be used in place of any or all of the prior art clamps 10. The clamp 22 primarily includes a tightening handle 24, a first clamp member 26 (in the lower position as shown in FIGS. 2-8, it being recognized that orientation of the clamp 22 may depend upon use as shown in FIG. 1) and a second clamp member 28 (in the upper position as shown in FIGS. 2-8). For ease of description, the first or lower clamp member 26 will be called a “frame” clamp and the second or upper clamp member 28 will be called a “shaft” clamp. Recognizing that the first clamp 26 may attach to a rod other than the support frame 18 and the second clamp 28 may attach to a rod other than a retractor shaft 16.

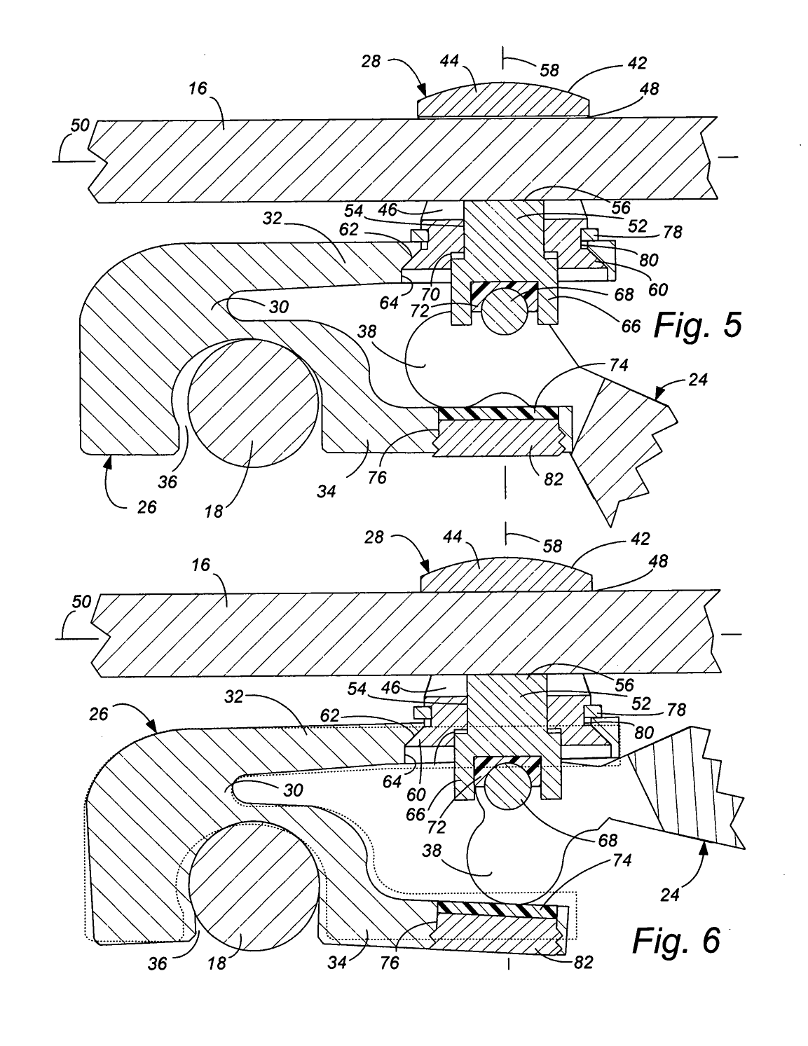

The frame clamp 26 may be a fulcrum clamp as generally disclosed in U.S. Pat. No. 5,727,899 and in application Ser. No. 10/664,195 filed Sep. 17, 2003, both incorporated by reference. The preferred fulcrum clamp 26 thus includes a fulcrum portion 30 extending between an upper leg portion 32 and a lower leg portion 34. As best illustrated in FIGS. 5 and 6, the fulcrum portion 30 allows the size of the frame clamp opening 36 to change based upon biasing the upper leg portion 32 away from the lower leg portion 34. A wedge or cam 38 positioned between the upper and lower leg portions 32, 34 is movable to force the upper and lower leg portions 32, 34 apart and causes the fulcrum portion 30 to flex. As the fulcrum portion 30 flexes, the frame clamp opening 36 constricts. When the frame clamp opening 36 constricts, the clamp 22 can frictionally attach onto the support frame 18. The frame clamp opening 36 extends longitudinally on the frame 18 for a sufficient distance to define a rod axis 40 and orientation of the frame 18 in the frame clamp opening 36. In the preferred embodiment, the frame clamp 26 is about 3/4 inch wide.

A significant advantage of using a fulcrum clamp for the frame clamp 26 is that it includes a clamping opening 36 which is open from below. The clamping opening 36 therefore permits the frame clamp 26 to be placed on the retractor support frame 18 without requiring threading the clamp body onto the support frame 18 and without moving any other surgical equipment that has been previously disposed upon the retractor support frame 18. The frame clamp 26 may be lightly and quickly snapped onto the support frame 18 at any substantially straight location desired by the surgeon. The frame clamp opening 36 is sized to mate with the cross-sectional size and shape of the support frame 18, such as a 1/2″ diameter cylindrical shape. In the loosened position, the frame clamp 26 permits substantially unimpeded longitudinal movement of the clamp 22 on the support frame 18, as well as substantially free rotation of the clamp 22 about the support frame axis 40.

Another significant advantage of using a fulcrum clamp is that the clamping force is easily made to be self-sustaining by use of a wedge or cam member 38 placing equal and opposite forces on the upper and lower leg portions 32, 34. Once the frame clamp 26 is closed to a tightened position, it does not require further application of force or holding by the surgical staff to remain in the tightened position. The preferred frame clamp cam 38 includes two lobes so it can provide a balanced force and for ease of manufacture and assembly.

A third significant advantage of using a fulcrum clamp is that the actuating mechanism can be positioned closely in to the support frame 18, and at the same elevation as the support frame 18. As will be appreciated throughout this discussion, the location of the handle 24 for the frame clamp 26 relative to the support frame 18 and relative to the retractor shaft 16 provides significant benefits during surgery.

The shaft clamp 28 is preferably also open for side or top attachment of the retractor shaft 16, such as the side attachment clamp shown. The shaft clamp 28 primarily includes a stanchion portion 42 having a top stanchion 44 extending rigidly from a side stanchion 46. The inside surfaces of the top stanchion 44 and side stanchion 46 are sized and shaped to mate with the retractor shaft 16. The shaft clamp opening 48 extends longitudinally on the shaft 16 for a sufficient distance to define a rod axis 50 and orientation of the shaft 16 in the shaft clamp opening 48. In the preferred embodiment, the shaft clamp 28 is about 1/2 inch wide.

As best shown in FIGS. 5 and 6, the shaft clamp 28 is activated by the same handle 24 as the frame clamp 26. To achieve the simultaneous tightening with a single handle 24, upward movement of the handle 24 not only causes the wedge 38 to increase separation between the upper and lower legs 32, 34, but also moves a pin 52 vertically upward to press the retractor shaft 16 against the top stanchion 44. The pin 52 translates or slides in a pin bore 54 in the bottom of the shaft clamp 28. The pin bore 54 intersects the shaft clamp opening 48, so the pin 52 can be biased against the outer profile of the shaft 16 by sliding the pin 52 in the bin bore 54.

A significant advantage of using a side snap clamp for the shaft clamp 28 is that it includes a clamping opening 48 which is open from above the support frame 18. The clamping opening 48 therefore permits the retractor shaft 16 to be placed into the side snap clamp without requiring threading of the shaft 16 through the shaft clamp opening 48. The shaft 16 may be lightly and quickly snapped into the shaft clamp 28 at any longitudinal location and angular orientation (i.e. either pivoted about the support frame 18 to a non-horizontal angle, or pivoted about a vertical axis as described below) desired by the surgeon. The shaft clamp opening 48 is sized to mate with the cross-sectional size and shape of the shaft 16, such as a ⅜″ diameter cylindrical shape. In the loosened position, the shaft clamp 28 permits substantially unimpeded longitudinal movement of the shaft 16 in the shaft clamp opening 48, as well as substantially free rotation of the shaft 16 about its axis 50 in the shaft clamp opening 48.

The upper end 56 of the pin 52 is shaped and oriented to mate with the outside surface of the shaft 16. In particular, the contact surface area of the sliding pin 52 should be half or more of the cross-sectional area of the sliding pin 52 measured perpendicular to the sliding direction. In the tightened position, a significant amount of force is transmitted between the pin 52 and the retractor shaft 16. By distributing this force over a significant surface area rather than a point contact, dimpling or other damage of the shaft 16 is avoided, making the retractor system more reusable over a longer life and more aesthetically acceptable as “looking like new”. For a ⅜″ diameter cylindrical shaft 16, the preferred pin 52 has a negative cylindrical contour 56 which contacts the shaft 16 over a surface area which extends about 0.35 inches longitudinally and for about 0.25 inches (about 60°) around the shaft axis 50, for a contact surface area of about 0.09 in².

The shaft clamp 28 is pivotable relative to the frame clamp 26 about a vertical axis 58. To achieve the pivoting feature, the shaft clamp 28 is attached to the frame clamp 26 through a rotatable attachment. After the clamp 22 is positioned on the support frame 18 and the shaft 16 is positioned in the shaft clamp 28 but before the handle 24 is moved from the loosened position to the tightened position, the shaft 16 is pivotable about the pivot axis 58. In most surgical procedures and as depicted in the figures, pivoting of the shaft 16 will take the shaft 16 through a shaft travel path which, in the preferred embodiment, is a substantially horizontal plane. The handle movement direction is at a substantial angle to the shaft travel path, preferably perpendicular to the horizontal shaft travel plane.

As shown in FIGS. 5 and 6, the preferred rotatable attachment has a frustroconical bottom flange 60 formed integrally with the side stanchion 46. The frustroconical bottom flange 60 mates with a frustroconical recess 62 in a bore 64 within the upper leg portion 32 of the frame clamp 26. The frustroconical nature of this mating relationship permits pivoting of the shaft clamp 28 relative to the frame clamp 26 so long as the handle 24 is in the loosened position, but frictionally prevents pivoting of the shaft clamp 28 relative to the frame clamp 26 once pressure is applied by the handle 24 in the tightened position.

The handle 24 is keyed to the shaft clamp 28 so the handle 24 moves with the shaft clamp 28 and controls the pivoting location of the shaft clamp 28 about the vertical axis 58. The preferred keying structure is through the tightening pin 52. A proximal end of the tightening pin 52 includes a non-circular yoke 66, and a cam pin 68 is fixed to the proximal end of the handle 24 and resides within the yoke 66. An opening 70 in the bottom of the stanchion portion 42 mates with and receives the non-circular yoke 66. The preferred non-circular yoke 66 has a rectangular horizontal cross-section. When the handle 24 is pivoted about the vertical axis 58, the cam pin 68 also pivots, causing both the non-circular yoke 66 and the keyed stanchion portion 42 to also pivot. After the clamp 22 is on the support frame 18 and while the handle 24 is still in the loosened position, the surgeon can easily select and adjust the orientation of the shaft clamp 28 in two ways: either by grasping the handle 24 and pivoting the handle 24 about the pivot axis 58, or by placing the retractor shaft 16 in the shaft clamp opening 48 and lightly pivoting the retractor shaft 16 about the pivot axis 58. Either way, the handle 24 always stays oriented in a vertical plane beneath the retractor shaft 16. If desired, the handle plane could be offset somewhat from the retractor shaft axis 50, preferably remaining at least parallel to the retractor shaft axis 50 if not aligned with the retractor shaft axis 50.

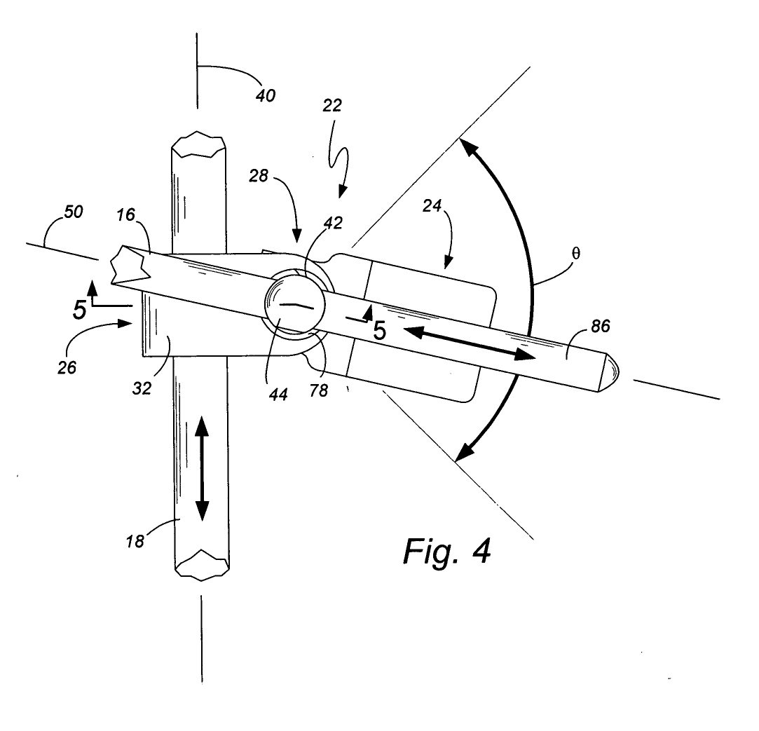

The preferred clamp 22 permits pivoting of the shaft 16 relative to the support frame 18 through an angle θ best shown in FIG. 4 before the handle 24 interferes with the frame clamp 26 on either side. The pivoting angle θ is symmetric relative to the bisecting plane of the frame clamp 26, so the entire clamp 22 retains a balanced appearance and feel. In the preferred embodiment, the pivoting angle θ is about 90°.

To aid in longevity of the clamp 22, the bearing surface between the cam pin 68 and the non-circular yoke 66 can be provided by a low friction insert 72. The bearing surface between the frame clamp cam 38 and the lower leg 34 of the frame clamp 26 can be provided by a washer 74. In the preferred embodiment, both the low friction insert 72 and the washer 74 are formed of a low friction polymer such as nylon???. The remaining components may be formed of an appropriately strong sterilizable metal, such as surgical stainless steel.

During assembly of the preferred clamp 22, the stanchion portion 42 is inserted into the bore 64 of the upper leg portion 32 from below. An opening 76 in the lower leg portion 34 may be provided for access to assist in machining of the upper leg portion 32 and to assist in placement of the stanchion portion 42 into the bore 64 from below. The same opening 76 may be used to position the pin 52 including the non-circular yoke 66 into its keyed opening 54 in the stanchion portion 42. The handle 24 is assembled in place from the side, with the cam pin 68 disposed in the yoke 66. Once in position, the stanchion portion 42 is then loosely secured to the frame clamp 26 with a snap ring 78. An annular groove 80 on the stanchion portion 42 is positioned above an upper surface of the upper leg portion 32. The snap ring 78 is disposed within the annular groove 80 and rotatably holds the stanchion portion 42 within the upper leg portion 32 of the frame clamp 26. Assembly is completed by disposing the washer 74 beneath the frame clamp cam 38 and tightening a bottom screw cap 82 in place. If desired, one or more springs (not shown) may be used, positioned anywhere between the bottom screw cap 82 , the washer 74, the frame cam 38, the shaft cam pin 68, the insert 72 and the yoke 66, to place a vertically oriented force on the assembly and thereby prevent any separation or rattling of parts which might otherwise occur if the dimensional tolerances on any of these parts are not strictly met.

The preferred clamp 22 accordingly permits a loosened attachment to both the support frame 18 and the retractor shaft 16 which has five degrees of freedom: the clamp 22 can be slid longitudinally on the support frame 18; the clamp 22 can be rotated about the longitudinal axis 40 of the support frame attachment portion; the shaft 16 can be pivoted about the vertical axis 58; the shaft 16 can be slid longitudinally in the shaft clamp 28; and the shaft 16 can be rotated about the longitudinal axis 50 of the shaft attachment portion. When the handle 24 is “thrown” or pivoted upward from the loosened position to its tightened position, all five of these degrees of freedom are secured. During tightening, both the shaft opening and the frame opening dimensions are slightly decreased to eliminate any rotation or translation of the shaft 16 and frame 18 relative to the clamp 22. At the same time, the frictional engagement of the mating frustroconical surfaces 60, 62 after tightening prevent further pivoting of the shaft clamp 28 relative to the frame clamp 26.

The linkage between the handle 24 and the frame clamp 26 and the shaft clamp 28 is fully operated between the fully loosened position and the fully tightened position by a pivoting of the handle 24 about the shaft cam pin 68 through a tightening throw range (φ In contrast to prior art devices, the entire tightening range (φ for the clamp 22 is targeted to conform. to the size and motion appropriate for a surgeon's hand, as best shown in FIG. 3. To tighten the clamp 22, the surgeon need only grasp the handle 24 with the surgeon's fingers and the shaft 16 with the surgeon's thumb on the same hand, and squeeze similar to operating a scissors. This scissors squeezing motion is very intuitive, as students have been taught to use scissors since kindergarten. Thus, the handle 24 has a grasping surface 84 on its bottom which is always aligned with and is generally facing away from the top biasing surface 86 of the shaft 16. In particular, the grasping surface 84 of the handle 24 should remain within about six inches or less from the top biasing surface 86 of the shaft 16. The handle 24 should move an entire distance of about five inches or less. The distal end of the handle 24 may extend slightly beyond the grasping surface 84, for a total throw displacement of the distal end of the handle 24 of about six inches or less. To enable the scissors action, the tightening throw should proceed through an angle φ of 90° or less. In the tightened position, the grasping surface 84 of the handle 24 positions a distal end of the handle 24 a distance d of from ½ to three inches from the top biasing surface 86, which enables a strong single handed grasping force to the fully tightened position. The preferred handle 24 extends for a length of about 3 inches, and pivots through a tightening throw range φ of about 60°. In the fully tightened position, the handle 24 extends substantially parallel to the shaft axis 50.

At this size, amount of pivoting and location of the pivot point 68, the grasping surface 84 of the handle 24 is 4 inches away from the top biasing surface 86 of the shaft 16 while in the loosened position, and is about 1 inch away from the top biasing surface 86 of the shaft 16 when in the tightened position. The handle/shaft combination thus act in conjunction to ergonomically fit the grasp of most surgeons' hands for a single handed, intuitive tightening operation.

Another feature achieved by the clamp 22 of the present invention is that the plane of handle movement is vertically upward. By orienting the pull direction of the handle 24 vertically upward for most placements of the clamp 22 assists in the wrist angle required of the surgeon to effect the squeezing motion required for tightening the clamp 22. That is, the surgeon is most commonly standing roughly in line and behind the retractor shaft 16. Just as when cutting a wide cloth a seamstress will lean over a flatly laid cloth and cut away from his or her body, so too the surgeon tightens the clamp 22 with a natural “cutting” orientation, fingers down and thumb up, similar to a handshake position. While the clamp 22 can be readily tightened with a wide variety of single handed orientations, the most common hand orientation naturally coincides with the most common clamp orientation and strongest grip orientation relative to the person tightening the clamp 22.

Orienting the pull direction upward also assists in avoiding displacement of the clamp 22 off the support frame 18 or movement of the support frame 18 during the typical tightening orientation. That is, the support frame 18 and the clamp 22 are both used with gravitational forces pulling them downward. A tightening force which is oriented downward combines with gravitational forces to more severely load the attachment of the frame clamp 26 to the frame 18 and to more severely load the supporting attachments further upstream on the support frame 18. The present invention commonly orients the tightening force upward, acting against gravity. By orienting the tightening force upward, there is less chance that the tightening force will pry the clamp 22 off the support frame 18 and less chance that the tightening force will disrupt the support frame 18 at its attachment to the post 22. The loosening force is oriented downward, but such loosening force is often applied after the clamp 22 and support frame 18 have served their purpose and during disassembly, when misalignment of the support frame 18 caused by the loosening force is less critical.

As best shown in FIGS. 4, 7 and 8, the handle 24 in the preferred clamp 22 is balanced relative to the handle movement plane, i.e., symmetrical about a vertical plane through the shaft axis 50. This balanced shape and position of the handle 24 further assists in the natural scissors action required to tighten the clamp 22.

As commonly desired, the clamp 22 is capable of being used with a vertical plane containing the shaft axis 50 being oriented perpendicular to the support frame axis 40. For instance, if the shaft/handle of the clamp 22 as shown in the plan view of FIG. 4 is pivoted to a three o'clock position, the entire clamp 22 is balanced and symmetrical about a bisecting vertical plane. When the tightening force is applied upward in this most common position, the tightening force is exactly balanced relative to the attachment of the frame clamp 26 to the support frame 18. A tightening force can be applied to the handle 24 which will not result in any twisting of either the left side or the right side of the frame clamp 26 off the support frame 18. By having a symmetrically-balanced, most-common position, the clamp 22 is less likely to twist off the support frame 18 during tightening.

Whether in the tightened position or in the loosened position, the handle 24 always resides vertically underneath the shaft 16. This position is best shown in FIG. 8, wherein the handle 24, even in the tightened position, fully resides within a “vertical shadow” σ defined by the horizontally extending shaft 16. In the preferred embodiment, the handle 24 fully resides within a 120° shadow a beneath the shaft 16. That is, the handle 24 resides in the area underneath two planes defined as being tangent to the horizontally oriented shaft 16 and inclined 30° from horizontal. This location is important because it naturally positions the shaft 16 to protect the handle 24 from unintentional bumping, and protects the distal end of the handle 24 from unintentionally catching upon something (a tube, a wire, a shirt sleeve, etc.) during surgery. The shaft 16 actually functions to prevent unintentional movement of the handle 24, particularly important so the handle 24 does not become unintentionally loosened during surgery. One preferred handle 24 is about an inch wide and has a tightened position spaced with a gap of about 0.2 inches beneath the ⅜″ diameter shaft 16, and thus resides within a vertical shadow a of the shaft 16 of about 60°. The corner of the handle 24 shown in FIG. 8 actually leaves a smaller gap than 0.2 inches, resulting in a slightly larger shadow angle σ immediately adjacent the shaft clamp 28, but still well within the 120° protective shadow angle guideline.

The same protective result could be alternative obtained by orienting the handle 24 such that it was maintained within a 120° shadow angle underneath the support frame 18. However, the retractor system 12 is typically assembled by placing the support frame 18 in place (usually, prior to making surgical incisions) prior to placement of the retractor shafts 16 (usually placed after making surgical incisions). Additionally, the support frame 18 is usually placed quite close to the patient's body, such as within the nearest six inches to the patient's body and sometimes touching the patient's body or garments. Accordingly, it is easier to place the retractor shafts 16 above the support frame 18 rather than below the support frame 18. With the retractor shaft 16 extending over the support frame 18, the most low profile, protected location for the handle 24 is aligned within the 120° shadow angle σ beneath the retractor shaft 16.

The typical placement of the support frame 18 around a surgical incision location with the retractor blade shafts 16 extending generally radially outward from the incision also. coincides with the throw of the handle 24 of the preferred clamp 22. That is, if the support frame 18 is positioned very close or in contact with a patient's body or garments around a surgical incision, the handle 24 will typically be positioned radially outward from the support frame 18 at a location that the patients' body angles away from the plane of the support frame 18. By locating the handle 24 so its tightening throw pivots the handle 24 about a horizontal axis (the axis of the shaft cam pin 68) which is roughly in the horizontal plane of the support frame 18, the surgeon can still place his or her fingers underneath the handle 24 (between the patient's body and the handle 24) for the tightening throw, and the entire throw of the handle 24 does not interfere in any way with the placement location of the support frame 18 relative to the incision.

The common orientation of the clamp 22 places both the shaft clamp 28 and the handle 24 outside the support frame 18, extending in the tightened position a total length of about five inches or less from the support frame 18. This places both the handle 24 and the shaft clamp 28 in the optimum position to avoid interference with the surgical arena defined inside the support frame 18, but still closely in to the support frame 18. In the preferred embodiment, the pivot axis 58 is within about an inch of the support frame axis 40. When the handle 24 is in the tightened position and aligned perpendicular to the support frame axis 40, the preferred embodiment still has a length of less than 4½ inches, with only about 0.3 inches of the fulcrum clamp 26 extending to the surgical arena side of the support frame 18. By placing both the shaft clamp 28 and the handle 24 on the same, outer side of the frame clamp 26, the entire clamp 22 fits in a small space and minimally intereferes with the surgical arena.

Thus it can be seen that the location and orientation of the handle 24 relative to the frame clamp 26 and relative to the shaft clamp 28 provide many advantages during the surgical procedure which are not provided by prior art clamps. While many linkage mechanisms can be used to translate tightening and loosening forces between the handle 24 and the frame clamp 26 and shaft clamp 28, the preferred linkage is cost effective, simple to manufacture and provides a very robust and easy to use clamp in an elegant design.

Although the present invention has been described with reference to preferred embodiments, workers skilled in the art will recognize that changes may be made in form and detail without departing from the spirit and scope of the invention. For instance, while the preferred embodiment includes two clamping members, certain aspects of the invention could be practiced with a single clamping member, such as integral with and/or permanently attached to either the support frame 18 or the retractor shaft 16. While a particular linkage is described which enables the handle 24 to simultaneously control both clamping members, it is recognized that many other types of linkages could be used while still obtaining the handle orientation benefits of the present invention.

1. A surgical clamp comprising:
a first clamp member having a first opening for receiving a first rod, the first opening changing dimensions to receive the first rod in a loosened first clamp position and in a tightened first clamp position, the first opening defining a first rod axis;
a second clamp member having a second opening for receiving a second rod, the second opening changing dimensions to receive the second rod in a loosened second clamp position and in a tightened second clamp position, the second clamp member being attached to the first clamp member;
a handle pivotable for moving the first clamp between its loosened first clamp position and its tightened first clamp position, wherein the handle has a total pivot throw between the loosened first clamp position and the tightened first clamp position of 90° or less.

2. The surgical clamp of claim 1, wherein pivoting of the handle moves the second clamp between its loosened first clamp position and its tightened first clamp position simultaneously with moving the first clamp between its loosened first clamp position and its tightened first clamp position.

3. The surgical clamp of claim 1, wherein the handle pivots in a handle plane, wherein the handle plane always substantially contains or is parallel to the first rod axis.

4. The surgical clamp of claim 1, wherein the second clamp member and the handle are both on the same side of the first clamp member.

5. The surgical clamp of claim 1, wherein the tightening of the handle positions a distal end of the handle from ½ to three inches from the first rod axis.

6. The surgical clamp of claim 1, wherein the total pivot throw of the handle moves a distal end of the handle a distance of six inches or less.

7. The surgical clamp of claim 1, wherein the first opening is open for receiving the first rod in a direction transverse to the first rod axis, wherein the second opening defines a second rod axis, and wherein the second opening is open for receiving the second rod in a direction transverse to the second rod axis.

8. A surgical scissors clamp tightenable with a single hand comprising:
a clamp member having an opening for receiving a rod, the opening changing dimensions to receive the rod in a loosened clamp position and in a tightened clamp position, with the loosened clamp position retaining the rod in the opening for substantially unimpeded longitudinal movement in the opening, the tightened clamp position frictionally restraining the rod against movement in the opening; and
a handle pivoting in a handle plane, pivoting of the handle moving the clamp. between its loosened clamp position and its tightened clamp position, wherein a complete throw of the handle from the loosened clamp position and the tightened clamp position can be accomplished by a single squeeze of a single hand.

9. The surgical scissors clamp of claim 8, wherein the clamp member defines a rod axis and wherein, when the first clamp is in the tightened clamp position, the handle is substantially parallel to the rod axis.

10. A surgical clamping system comprising:
a support frame, at least a connection portion of the support frame defining an axis and extending generally horizontally;
a shaft, at least a connection portion of the shaft defining an axis and extending generally horizontally;
a clamp member for connecting the connection portion of the shaft to the connection portion of the support frame, the clamp member having a tightened position wherein the shaft is frictionally fixed relative to the support frame and a loosened position wherein the shaft is moveable relative to the support frame; and
a handle for moving the clamp member between the loosened position and the tightened position, wherein, when the handle is in the tightened position, the handle is positioned within a 120° shadow beneath one of the connection portion of the support frame and the connection portion of the shaft.

11. The surgical clamping system of claim 10, wherein the handle is raised upward from the loosened position to the tightened position.

12. The surgical clamping system of claim 10, wherein the clamp member is in the tightened clamp position, the handle is substantially parallel to one of the connection portion of the support frame and the connection portion of the shaft.

13. A surgical clamping system comprising:
a support frame, at least a connection portion of the support frame defining an axis and extending generally horizontally;
a shaft, at least a connection portion of the shaft defining an axis and extending, generally horizontally;
a clamp member for connecting the connection portion of the shaft to the connection portion of the support frame, the clamp member having a tightened position wherein the shaft is frictionally fixed relative to the support frame and a loosened position wherein the shaft is moveable relative to the support frame; and
a handle for moving the clamp member between the loosened position and the tightened position, wherein the handle is raised upward from the loosened position to the tightened position.

14. A clamp comprising:
a support frame attachment structure;
a clamp member having an opening for receiving a rod, the opening changing dimensions to receive the rod in a loosened clamp position and in a tightened clamp position, the opening defining a rod axis, the clamp member when in a loosened clamp position being pivotally connected to the support frame attachment structure, such that movement of the pivotal connection causes the rod axis to define a rod movement travel path; and
a handle pivotable in a handle plane disposed at an angle to the rod movement travel path, pivoting of the handle moving the first clamp between its loosened first clamp position and its tightened first clamp position, wherein the handle plane always substantially contains or is parallel to the rod axis regardless of movement of the pivotal connection through the rod movement travel path.

15. A surgical clamp system comprising:
a rod extending longitudinally having an exterior profile
a clamp member having a rod opening for receiving the rod, the clamp member having a pin bore intersecting the rod opening;
a sliding pin disposed for longitudinal sliding in the pin bore such that a biasing end of the sliding pin can bias the rod within the clamp member to tighten the rod within the clamp member, the biasing end of the sliding pin having a contour which mates with the exterior profile of the rod across a contact surface area; and
a handle for sliding the sliding pin longitudinally within the pin bore.

16. The surgical clamp of claim 15, wherein the sliding pin has a cross-sectional area measured within and perpendicular to the sliding pin bore, wherein the contact surface area of the sliding pin is at least half of the cross-sectional area of the sliding pin.

17. The surgical clamp of claim 15, wherein the exterior profile of the rod is cylindrical, and wherein the biasing end of the sliding pin has a negative cylindrical contour.

18. A surgical clamp comprising:
a first clamp member having a support frame opening for receiving a support frame rod, the support frame opening changing dimensions to receive the support frame rod in a loosened first clamp position and in a tightened first clamp position, the support frame opening defining a support frame rod axis and defining a bisecting plane perpendicular to the support frame rod axis halfway along the length of contact between the support frame rod and the first clamp member;
a second clamp member having a tool opening for receiving a tool rod at a position over the support frame rod axis, the second opening changing dimensions to receive the tool rod in a loosened second clamp position and in a tightened second clamp position, the second clamp member being attached to the first clamp member; and
a handle pivotable for moving the first clamp between its loosened first clamp position and its tightened first clamp position, wherein the handle has a longitudinal axis capable of pivoting within the bisecting plane.

19. The surgical clamp of claim 18, wherein with the longitudinal axis within the bisecting plane, the handle is symmetrical about the bisecting plane.
